(19) United States
(12) Patent Application Publication
(10) Pub. No.: US 2024/0206903 A1
(43) Pub. Date: Jun. 27, 2024

(54) MEDICAL SHAVER WITH MULTI-BLADE

(71) Applicant: KARL STORZ SE & Co. KG, TUTTLINGEN (DE)

(72) Inventor: Vernon HOPKINS, Worcester, MA (US)

(21) Appl. No.: 18/085,964

(22) Filed: Dec. 21, 2022

(51) Int. Cl. A61B 17/32; A61B 17/3207

(52) U.S. Cl. CPC A61B 17/32002; A61B 17/320758

(57) ABSTRACT

A multi-blade for a medical shaver includes an outer blade and an inner blade. The outer blade includes a keyhole through which at least a portion of the inner blade is exposed. The keyhole includes a first set of corresponding cutting edges on a first side of the outer blade and the inner blade, and a second set of corresponding cutting edges on a second side of the outer blade and the inner blade. Rotation of the inner blade in a first direction removes a first amount of tissue from a patient with the first set of cutting edges. Rotation of the inner blade in a second, different direction removes a second, different amount of tissue from the patient with the second set of cutting edges.

### BACKGROUND

The present disclosure is generally directed to medical shavers and, in particular, toward a multi-blade that is either fixed to or detachable from a shaver handpiece.

Select medical procedures such as arthroplasty may require the use of a medical shaver. For example, the medical procedure may be an endoscopic procedure. The medical shaver includes a shaver handpiece and a blade with a single cutting surface. The medical procedure may require one or several types of cuts to remove tissue (e.g., soft tissue such as cartilage, osseous tissue such as bone, or the like) in desired quantities. Due to the single cutting surfaces, where several types of blades are used, known techniques involve using multiple fixed-blade medical shavers and/or a medical shaver with multiple blade attachments. Swapping the known multiple fixed-blade medical shavers and/or the known multiple blade attachments may result in increased contamination of the surgical environment, as the location of an increased number of clean and used devices need to be monitored.

In addition, swapping between the known multiple fixed-blade medical shavers and/or the known multiple blade attachments requires additional procedure time and material cost for the medical procedure. The known fixed-blade multiple medical shavers and/or the known multiple blade attachments also require additional coordination between operators and assistants, and requires additional space in the surgical environment (e.g., on a surgical prep tray, or the like).

Further, known fixed-blade medical shavers and/or known blade attachments for a medical shaver may be disposable. Where the medical procedure requires several types of cuts, a similar number of fixed-blade medical shavers and/or blade attachments are required, resulting in increased disposable medical waste.

As such, it would be beneficial to provide a medical shaver which addresses the known issues, above.

### BRIEF SUMMARY

The present disclosure is directed to a medical shaver with a multi-blade that is either fixed to or attachable to/detachable from a shaver handpiece. In particular, the present disclosure is directed to a medical shaver with a multi-blade, where the multi-blade includes a keyhole with multiple cutting types or edges at a distal end of the multi-blade.

The multi-blade includes an inner blade or interior shaft and an outer blade or exterior housing. The outer blade defines a hollow cavity in which the inner blade is inserted. The hollow cavity also allows for the extraction of tissue (e.g., soft tissue such as cartilage, osseous tissue such as bone, or the like) from a surgical site through the outer blade or exterior housing, following the removal of the tissue with the various cutting types or edges at the surgical site.

The inner blade is capable of a rotational motion within the outer blade. For example, the outer blade may be fixed in position or is capable of being manually rotated relative to the shaver handpiece to which the outer blade is coupled, and the inner blade is rotatable relative to the shaver handpiece. Rotation of the inner blade in a first direction results in a first type of cut, and rotation of the inner blade in a second direction results in a second type of cut. The first type of cut may be different from the first type of cut.

In one non-limiting example, the first type of cut is an aggressive cut caused by a more-coarse cutting pattern with fewer teeth on a first set of proximate edges of the outer blade and the inner blade located on a first side of the multi-blade. The second type of cut is a smooth cut caused by a less-coarse cutting pattern with more teeth on a second side of the multi-blade on a second set of proximate edges of the outer blade and the inner blade located on a second side of the multi-blade. In this example, operating time and cost may be reduced due to the multi-blade approach.

In another non-limiting example, the inner blade includes the same cutting type or edge on both sides, whereas the outer blade includes different cutting types or edges on respective sides. Here, operating time may be increased from the above example, but manufacturing costs (e.g., for the inner blade) may be reduced.

In another non-limiting example, the outer blade includes the same cutting type or edge on both sides, whereas the inner blade includes different cutting types or edges on respective sides. Again, operating time may be increased from the first example, but manufacturing costs (e.g., for the outer blade) may be reduced.

In this regard, the medical shaver of the present disclosure improves upon or solves many problems that are inherent with medical shavers currently available on the market. The medical shaver of the present disclosure is more user-friendly and less complex than known medical shavers, not requiring the swapping of fixed-blade medical shaver and/or attachment blades where multiple type of cuts are necessary during the medical procedure. The medical shaver of the present disclosure addresses issues related to one or more of operational complexity, operational time, operational cost, manufacturing cost, and/or possibility of contamination found to be both common and problematic with known medical shavers. The medical shaver of the present disclosure offers improvements over known medical shavers in many areas such as operational complexity, operational time, operational cost, manufacturing cost, and/or possibility of contamination.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages are described herein and will be apparent to those skilled in the art upon consideration of the following Detailed Description and in view of the figures.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

It should be understood that the drawings are not necessarily to scale. In certain instances, details that are not necessary for an understanding of the disclosure or that render other details difficult to perceive may have been omitted. It should be understood, of course, that the disclosure is not necessarily limited to the particular embodiments illustrated herein.

FIG. 1A shows a perspective view of a first embodiment of a medical shaver with a multi-blade, in accordance with aspects of the present disclosure;

FIG. 1B shows a perspective view of an expanded sectional view including a portion of the multi-blade of FIG. 1A, in accordance with aspects of the present disclosure;

FIG. 1C shows a left elevation view of the portion of the multi-blade of FIG. 1B, in accordance with aspects of the present disclosure;

FIG. 1D shows a right elevation view of the portion of the multi-blade of FIG. 1B, in accordance with aspects of the present disclosure;

FIG. 1E shows a top plan view of the portion of the multi-blade of FIG. 1B, in accordance with aspects of the present disclosure;

FIG. 1F shows a bottom plan view of the portion of the multi-blade of FIG. 1B, in accordance with aspects of the present disclosure;

FIG. 1G shows a front elevation view of the portion of the multi-blade of FIG. 1B, in accordance with aspects of the present disclosure;

FIG. 1H shows a rear elevation view of the portion of the multi-blade of FIG. 1B, in accordance with aspects of the present disclosure;

FIG. 1I shows a perspective view of a second embodiment of a medical shaver with a multi-blade, in accordance with aspects of the present disclosure;

FIG. 1J shows a perspective view of an expanded sectional view including a portion of the multi-blade of FIG. 1I, in accordance with aspects of the present disclosure;

FIG. 1K shows a left elevation view of the portion of the multi-blade of FIG. 1J, in accordance with aspects of the present disclosure;

FIG. 1L shows a right elevation view of the portion of the multi-blade of FIG. 1J, in accordance with aspects of the present disclosure;

FIG. 1M shows a top plan view of the portion of the multi-blade of FIG. 1J, in accordance with aspects of the present disclosure;

FIG. 1N shows a bottom plan view of the portion of the multi-blade of FIG. 1J, in accordance with aspects of the present disclosure;

FIG. 1O shows a front elevation view of the portion of the multi-blade of FIG. 1J, in accordance with aspects of the present disclosure;

FIG. 1P shows a rear elevation view of the portion of the multi-blade of FIG. 1J, in accordance with aspects of the present disclosure;

FIG. 2 shows a perspective view of a distal end of a multi-blade for a medical shaver, in accordance with aspects of the present disclosure;

FIG. 3 shows a perspective view of a distal end of a multi-blade for a medical shaver, in accordance with aspects of the present disclosure;

FIG. 4 shows a first perspective view of a distal end of a multi-blade for a medical shaver, in accordance with aspects of the present disclosure;

FIG. 5 shows a second perspective view of the distal end of the multi-blade of FIG. 4;

FIG. 6 shows a first perspective view of a distal end of a multi-blade for a medical shaver, in accordance with aspects of the present disclosure;

FIG. 7 shows a second perspective view of the distal end of the multi-blade of FIG. 6;

FIG. 8 shows a first perspective view of a distal end of a multi-blade for a medical shaver, in accordance with aspects of the present disclosure;

FIG. 9 shows a second perspective view of the distal end of the multi-blade of FIG. 8;

FIG. 10 shows a first perspective view of a distal end of a multi-blade for a medical shaver, in accordance with aspects of the present disclosure;

FIG. 11 shows a second perspective view of the distal end of the multi-blade of FIG. 10;

FIG. 12 shows a first perspective view of a distal end of a multi-blade for a medical shaver, in accordance with aspects of the present disclosure;

FIG. 13 shows a second perspective view of the distal end of the multi-blade of FIG. 12; and

FIG. 14 shows a medical shaver system including a control unit and a medical shaver with a multi-blade, in accordance with aspects of the present disclosure.

### DETAILED DESCRIPTION

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The ensuing description provides embodiments only, and is not intended to limit the scope, applicability, or configuration of the claims. Rather, the ensuing description will provide those skilled in the art with an enabling description for implementing the described embodiments. It being understood that various changes may be made in the function and arrangement of elements without departing from the spirit and scope of the appended claims.

Various aspects of the present disclosure will be described herein with reference to drawings that may be schematic illustrations of idealized configurations.

Known medical shavers have issues related to one or more of operational complexity, operational time, operational cost, manufacturing cost, and/or possibility of contamination found to be both common and problematic. Known medical shavers include single-blade configurations that require swapping of blade attachments and/or entire shaver handpieces where multiple types of cuts are necessary.

It is with respect to the above issues and other problems that the embodiments presented herein were contemplated.

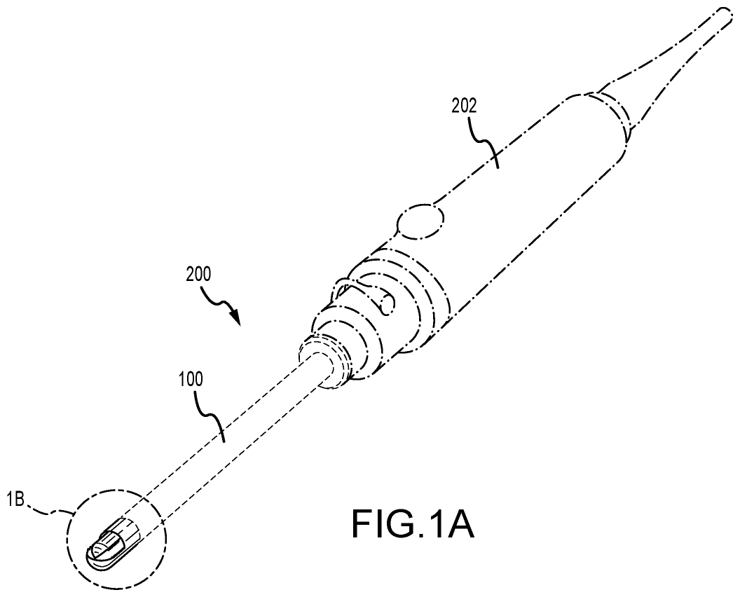

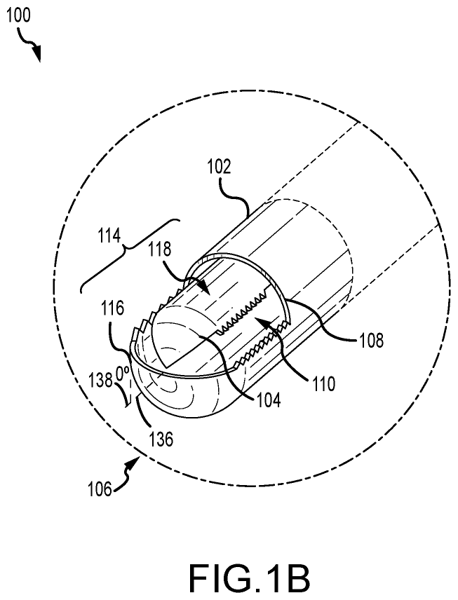

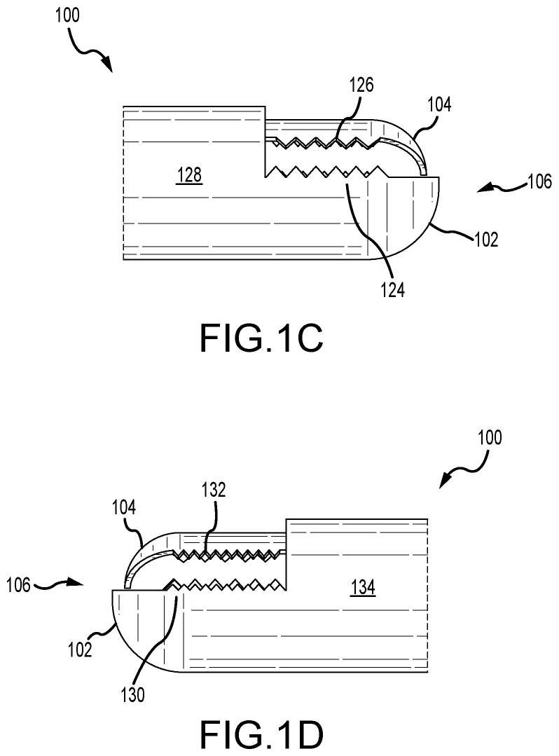

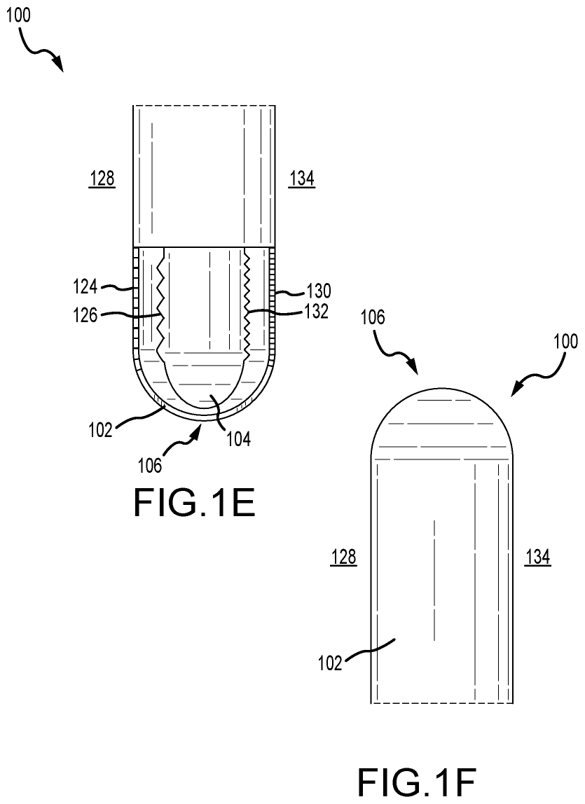

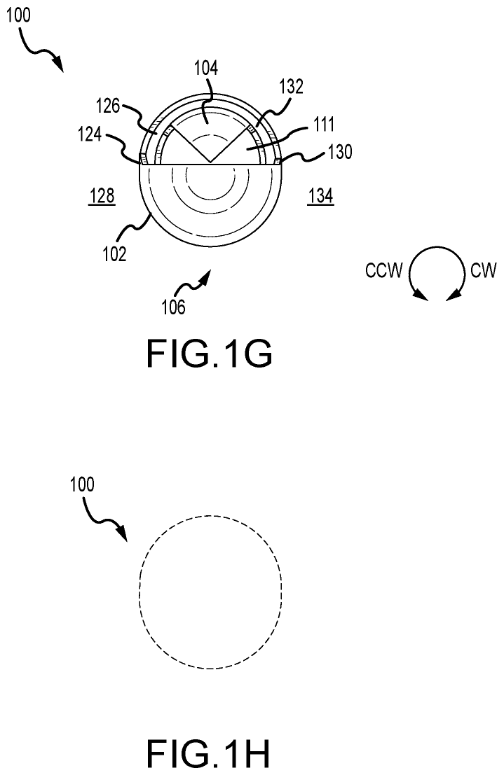

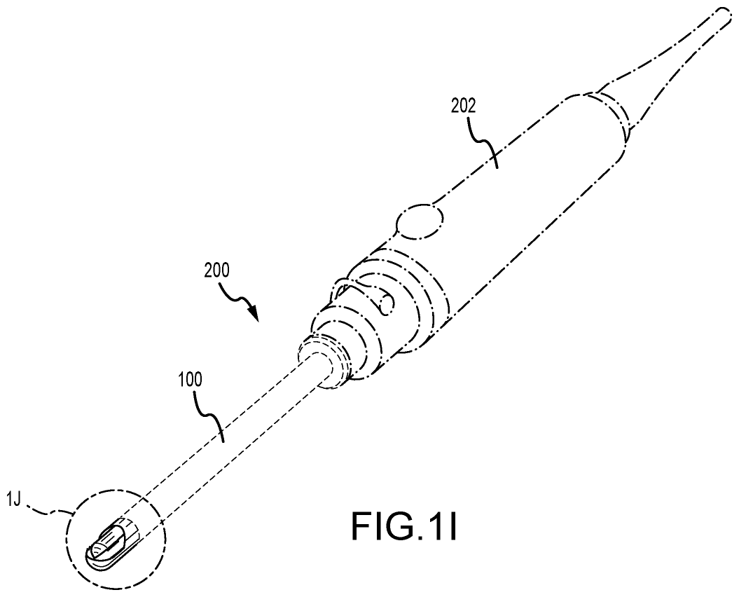

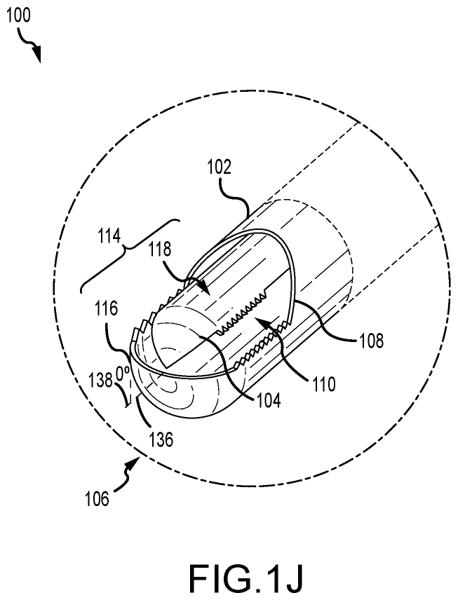

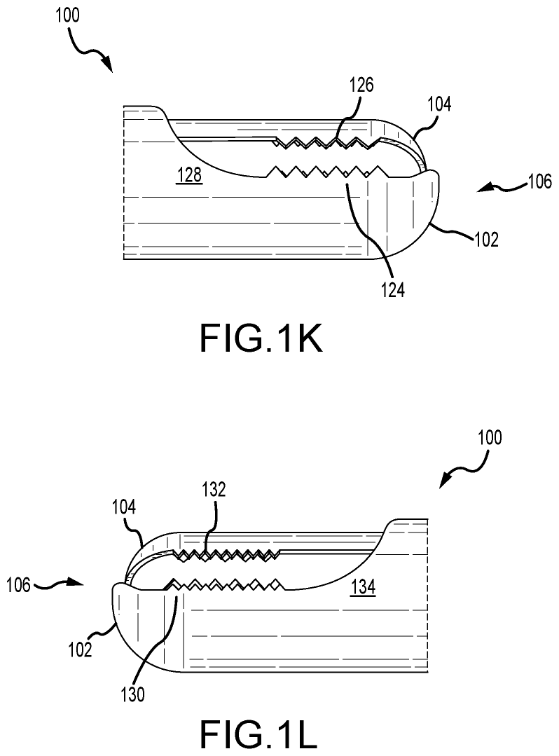

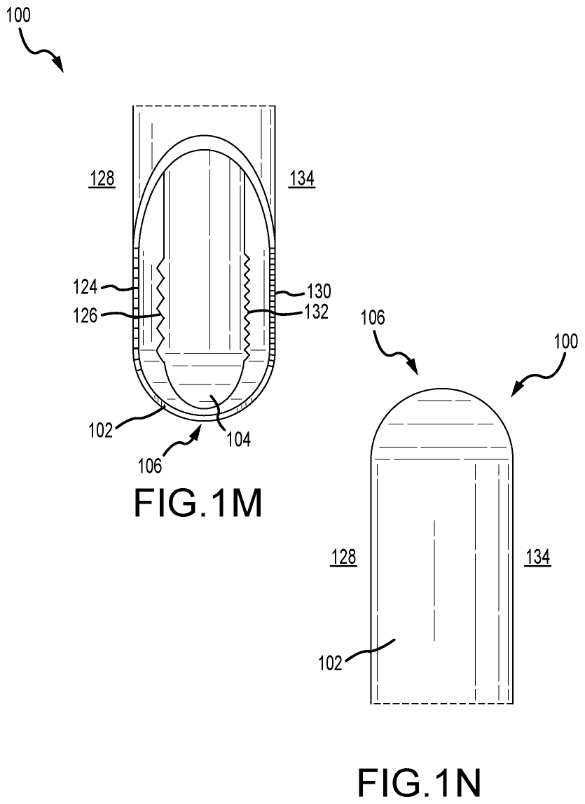

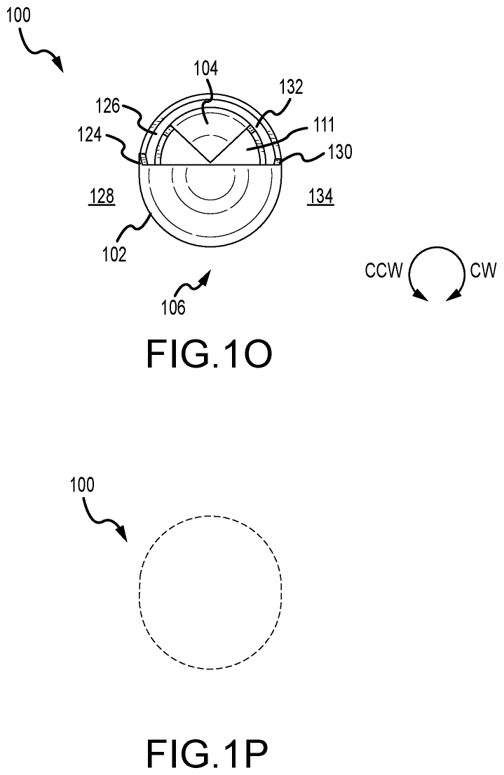

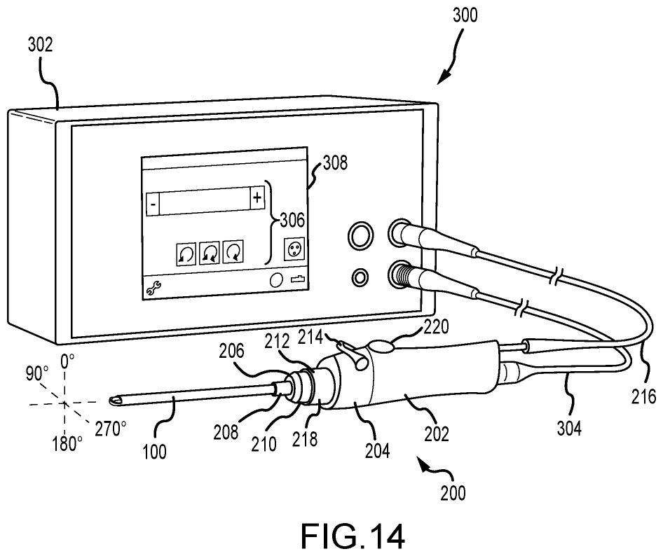

FIGS. 1A-14 generally show exemplary views of a multi-blade 100, in accordance with aspects of the present disclosure. In general, FIGS. 1A, 1I, and 14 illustrate the multi-blade 100 with an outer blade or exterior housing 102 and an inner blade or interior shaft 104, with exemplary views of an expanded sectional view of the multi-blade 100 in FIGS. 1B-1H, 1J-1P, 2, 3, and 4-13. In addition, FIGS. 1A, 1I, and 14 illustrate a medical shaver 200 including the multi-blade 100. Further, FIG. 14 illustrates a control system 302 in communication with the medical shaver 200 and the multi-blade 100.

Referring now generally to FIGS. 1A-1H, 1I-1P, 2, and 3, the multi-blade 100 includes a distal end 106. The multi-blade 100 includes the outer blade or exterior housing 102. In some aspects, a working diameter of the multi-blade 100 (e.g., an outer or exterior diameter of the outer blade 102) ranges from 2.5 millimeters (mm) (0.098 inches (in)) to 6.5 mm (0.256 in). The outer blade 102 includes one or more walls 108 that define an interior cavity 110.

The multi-blade 100 includes the inner blade or interior shaft 104. The inner blade 104 is insertable into the interior cavity 110. In some aspects, there is sufficient cavity space surrounding the inner blade 104 such that tissue (e.g., soft tissue such as cartilage, osseous tissue such as bone, or the like) cut during a surgical procedure may be removed through the interior cavity 110 of the outer blade 102. For example, the tissue may be removed from the distal end 106 to a proximal end of the multi-blade 100 along a longitudinal axis of the multi-blade 100 via a suction force applied through the multi-blade 100 (e.g., via an interior cavity 111 defined within the inner blade 104 and/or via the interior cavity 110) by the medical shaver 200, as described in detail further herein.

In general, the outer blade 102 and/or the inner blade 104 may be any three-dimensional shape having a known two-dimensional cross-section. In one instance, an exemplary outer blade 102 is cylindrical with a substantially circular cross-section for at least a portion of the length of the outer blade 102 between the distal end 106 and the proximal end of the multi-blade 100. In another instance, an exemplary inner blade 104 is a curved blade with a cross-section that forms an arc length for at least a portion of the length of the outer blade 102 between the distal end 106 and the proximal end of the multi-blade 100. In select configurations, the inner blade 104 transitions from a curved blade to a cylinder that is hollow or solid at some predefined distance from a distal tip along the length of the inner blade 104. In one non-limiting example, at least a portion of the inner blade 104 is a cylinder that is hollow and defines the interior cavity 111. In this example, a suction force may be applied through the interior cavity 111 defined by the inner blade 104 (e.g., as illustrated in FIG. 1G) to remove cut tissue from the surgical site via passage through the medical shaver 200. In other configurations, the inner blade 104 is a curved blade along approximately the entire length of the inner blade 104 (e.g., up to the point of engagement in a blade end 208 at a proximal end of the multi-blade 100, as described in detail further herein).

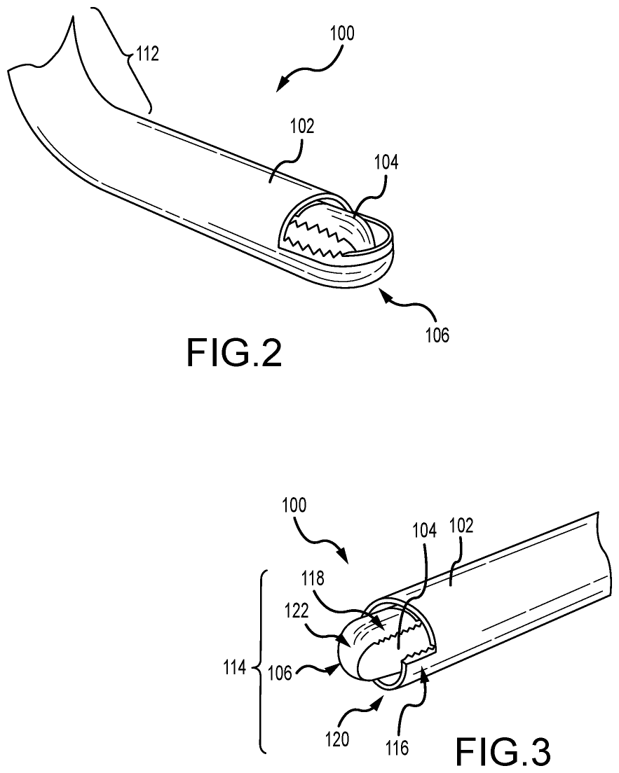

In some aspects, the multi-blade 100 is substantially linear. In other aspects, the multi-blade 100 includes one or more bends or bent sections 112 (e.g., the multi-blade 100 is curved or has curved sections). For example, as illustrated in FIG. 2, the one or more bends 112 may be proximate to the distal end 106 of the multi-blade 100. It is noted the one or more bends 112 may allow for the insertion of the multi-blade 100 at an angle relative to an incision or opening within a patient and the interior structure e.g., soft tissue such as cartilage, osseous tissue such as bone, or the like) to be cut with the multi-blade 100.

The distal end 106 of the multi-blade 100 includes a keyhole 114 in the outer blade 102, through which the inner blade 104 is at least partially exposed or protrudes along at least a portion of a length of the inner blade 104. As illustrated in a first embodiment of the multi-blade 100 in FIGS. 1A-1H, the keyhole 114 may include non-tapered sidewalls that end at a same distance from the distal tip for an entire arc length defining the keyhole 114. As illustrated in a second embodiment of the multi-blade 100 in FIGS. 1I-1P, the keyhole 114 is a tapered design that comes to a point a select distance from the distal tip of the multi-blade 100. It is noted that the first embodiment in FIGS. 1A-1H and the second embodiment in FIGS. 1I-1P are otherwise identical, except for the tapering of the keyhole 114.

In some aspects, the inner blade 104 is partially exposed through the keyhole 114, such that the keyhole 114 may include an exposure section 116 and the inner blade 104 includes an exposed portion 118. Here, the distal end 106 of the multi-blade 100 operates similar to a cutter or resector. The keyhole 114 may include an arc length that ranges from 1 degree to 359 degrees about an outer circumference of the outer blade 102, and preferably from 1 to 180 degrees. Where the keyhole 114 is tapered, as illustrated in FIGS. 1I-1P, the arc length defining the keyhole 114 may change in angle from the point the select distance from the distal tip of the multi-blade 100 to a final arc length (e.g., 180 degrees, or the like). In one instance, the arc length may be approximately 180 degrees about the outer circumference of the outer blade 102. It is noted the keyhole 114 may be contoured or stepped about the outer circumference of the outer blade 102 along the length of the keyhole 114, such that angle corresponding to the arc length of the keyhole 114 differs along the length of the keyhole 114.

In other aspects, the inner blade 104 protrudes or is fully exposed for at least a portion of a length of the inner blade 104 at the distal end 106, such as where the inner blade 104 fully protrudes from the outer blade 102 for at least a portion of the length of the inner blade 104. Here, the distal end 106 of the multi-blade 100 may operate similar to a burr, instead of cutting or resecting. The keyhole 114 may include a protrusion section 120 and the inner blade 104 may include a protruding section 122.

In some non-limiting examples, such as those illustrated in FIG. 3, the inner blade 104 may include a protrusion section 120 that transitions into an exposure section 116 along the length of the keyhole 114, such that the inner blade 104 includes both an exposed portion 118 and a protruding portion 122. It is contemplated, however, the keyhole 114 may only include the protrusion section 120 and the inner blade 104 may include only a protruding section 122, such that the outer blade 102 is effectively cut off at the distal end 106 of the multi-blade 100 and the inner blade 104 extends a select distance beyond the cut-off, without departing from the scope of the present disclosure.

The distal end 106 of the multi-blade 100 includes one or more cutting edges. In some aspects, the multi-blade 100 includes a first cutting edge 124 of the outer blade 102 and a corresponding first cutting edge 126 of the inner blade 104 on a first side 128 of the distal end 106. In other aspects, the multi-blade 100 additionally or alternatively includes a second cutting edge 130 of the outer blade 102 and a corresponding a second cutting edge 132 of the inner blade 104 on a second side 134 of the distal end 106. It is noted the first side 128 and the second side 134 of the distal end 106 may be considered as dependent on the angle defining the arc length of the keyhole 114 about the outer circumference of the outer blade 102. In this regard, the first side 128 and the second side 134 of the distal end 106 may be approximately 180 degrees apart, less than 180 degrees apart, or greater than 180 degrees apart.

The cutting edges of the multi-blade 100 include one or more cutting types or patterns. For example, the cutting edges of the multi-blade 100 may include, but are not limited to, a saw blade-type edge with an aggressive cut (e.g., a more-coarse cutting pattern with fewer teeth) for removing large amounts of tissue, a smooth cut (e.g., a less-coarse cutting pattern with more teeth) for removing small amounts of tissue, variations on the aggressive cut, variations on the smooth cut, or a serrated cut. By way of another example, the cutting edges of the multi-blade 100 may alternatively or additionally include a knife blade-type edge (e.g., with no teeth) with symmetric or asymmetric, single or double, surfaces. For instance, the knife blade-type surfaces may be flat, chisel, convex, compound bevel, concave, or hollow surfaces, or a combination thereof. It is noted that any combination of cutting edges of the multi-blade 100 is possible, without departing from the scope of the present disclosure. In addition, it is noted that the selected edge or combination of cutting edges of the multi-blade 100 may be dependent on the surgical procedure being performed. In this regard, the multi-blade 100 is configurable with any selected edge or combination of cutting edges during manufacture to provide the most benefit to the surgeon performing the surgical procedure.

The cutting edges 124, 130 are formed axially along a length of the outer blade 102. In some aspects, the cutting edges 124, 130 remain at the same angle relative to a defined 0 degrees on the outer blade 102 for the length of the cutting edges 124, 130, being parallel or approximately parallel to a longitudinal axis 136 of the outer blade 102 (e.g., as depicted in FIG. 1B). In other aspects, at least one of the cutting edges 124, 130 is configured to at least partially wrap about an outer circumference of the outer blade 102 while extending axially along the length of the outer blade 102, increasing or decreasing at an angle relative to the defined 0 degrees on the outer blade 102.

The cutting edges 126, 132 are formed axially along a length of the inner blade 104. In some aspects, the cutting edges 126, 132 remain at the same angle relative to a defined 0 degrees on the inner blade 104 for the length of the cutting edges 126, 132, being parallel or approximately parallel to a longitudinal axis 138 of the inner blade 104 (e.g., as depicted in FIG. 1B). In other aspects, at least one of the cutting edges 126, 132 is configured to at least partially wrap about an outer circumference of the inner blade 104 while extending axially along the length of the inner blade 104, increasing or decreasing at an angle relative to the defined 0 degrees on the inner blade 104.

It is noted the longitudinal axis 138 of the inner blade 104 may be parallel or substantially parallel to the longitudinal axis 136 of the outer blade 102. In addition, it is noted the longitudinal axis 138 of the inner blade 104 may be coaxial with the longitudinal axis 136 of the outer blade 102, or the longitudinal axis 138 of the inner blade 104 may be offset from the longitudinal axis 136 of the outer blade 102, without departing from the scope of the present disclosure.

It should be understood that portions of the multi-blade 100 and/or the medical shaver 200 may not form part of the claimed invention or design but rather is only a portion of an environment in which the claimed invention or design operates. In one non-limiting example, one or more aspects of the multi-blade 100, the medical shaver 200, and/or the control system 302 may not form part of the claimed invention or design but rather is only a portion of an environment surrounding the claimed invention or design and/or a portion of an environment in which the claimed invention or design operates. In one instance, at least a portion of the length of outer blade 102 (and inner blade 104, although not shown) of the multi-blade 100 may not form part of the claimed invention or design but rather is only a portion of an environment surrounding the claimed invention or design and/or a portion of an environment in which the claimed invention or design operates. It is noted a non-limiting example of the environment is depicted by the broken-line of the multi-blade 100 in FIGS. 1A-1H and FIGS. 1I-1P. In addition, it is noted a non-limiting example of the environment is depicted by the dot-dash broken line of the medical shaver 200 in FIGS. 1A and 1I. Therefore, the description through the present disclosure should not be interpreted as a limitation on the scope of the present disclosure but merely an illustration.

FIGS. 2-3 shows exemplary views of the distal end 106 of the multi-blade 100, in accordance with aspects of the present disclosure. As shown in FIG. 2, the multi-blade 100 may include the one or more bends 112 proximate to the distal end 106. As shown in FIG. 3, the multi-blade 100 may include both the protrusion section 120 and the exposure section 116 in the outer blade 102 at the distal end 106, along with the corresponding protruding portion 122 and the exposed portion 118 of the inner blade 104 (e.g., as opposed to only the exposure section 116 at the distal end 106, as shown in FIGS. 1A-1H, 1I-1P, 2, and 4-13).

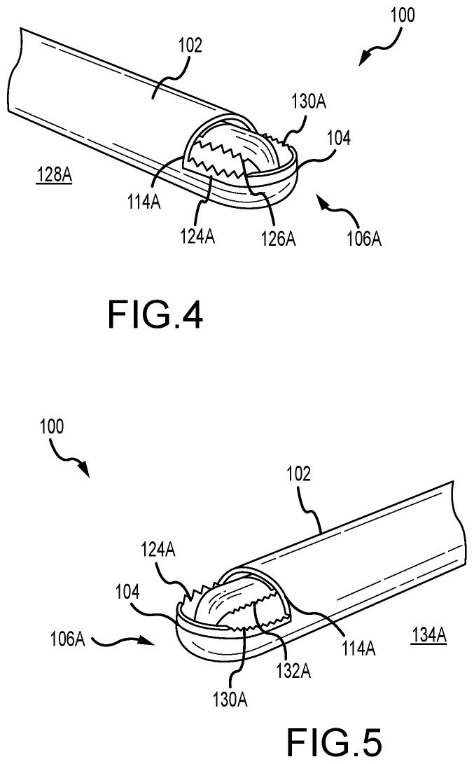

Exemplary views of a distal end 106A-106E and a keyhole 114A-114E are illustrated in FIGS. 4-13. It is noted that the exemplary distal ends 106A-106E and the keyholes 114A-114E of the multi-blade 100 as shown in FIGS. 4-13 have interchangeable aspects, unless otherwise noted in the specific respective descriptions of the distal ends 106A-106E and the keyholes 114A-114E of the multi-blade 100 herein. In addition, it is noted that the aspects described above with respect to FIGS. 1A-1H, 1I-1P, 2, and 3 should be understood as being applicable to FIGS. 4-13, unless otherwise noted in the specific respective descriptions of the distal ends 106A-106E and the keyholes 114A-114E of the multi-blade 100 herein.

Although not shown, it should be understood the non-tapered sidewalls illustrated in FIGS. 2-14 that end at the same distance from the distal tip for the entire arc length defining the keyhole 114 (e.g., similar to the first embodiment of the multi-blade 100 illustrated in FIGS. 1A-1H) may instead be a tapered design that comes to a point a select distance from the distal tip of the multi-blade 100 (e.g., similar to the second embodiment of the multi-blade 100 illustrated in FIGS. 1I-1P), without departing from the scope of the present disclosure.

Although not shown, it should be understood that a defined 0 degrees in FIGS. 4-5, 6-7, 8-9, 10-11, 12-13 would be similar to the defined 0 degrees based on the longitudinal axes 136, 138 as described above and depicted in FIG. 1B, without departing from the scope of the present disclosure.

FIGS. 4-5 shows exemplary views of the distal end 106A and the keyhole 114A of the multi-blade 100, in accordance with aspects of the present disclosure. In FIGS. 4-5, the first side 128A of the distal end 106A includes aggressive cutting edges 124A, 126A. In addition, the second side 134A of the distal end 106A includes smooth cutting edges 130A, 132A.

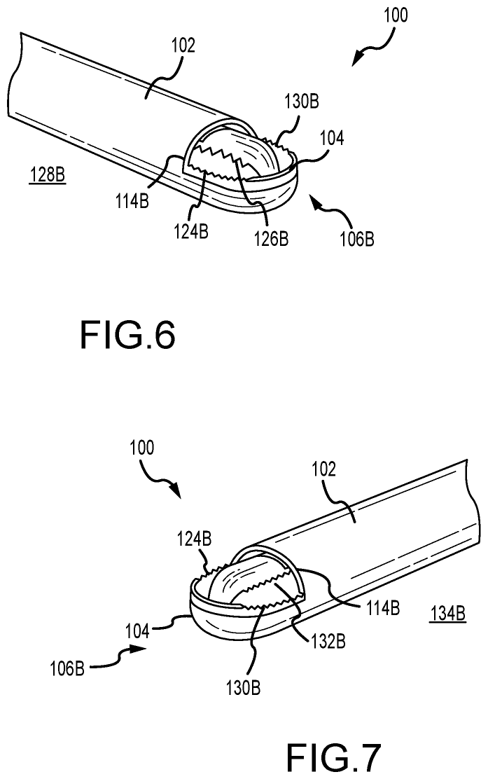

FIGS. 6-7 shows exemplary views of the distal end 106B and the keyhole 114B of the multi-blade 100, in accordance with aspects of the present disclosure. In FIGS. 6-7, the first side 128B of the distal end 106B includes a smooth cutting edge 124B for the outer blade 102 and an aggressive cutting edge 126B for the inner blade 104. In addition, the second side 134B of the distal end 106B includes smooth cutting edges 130B, 132B.

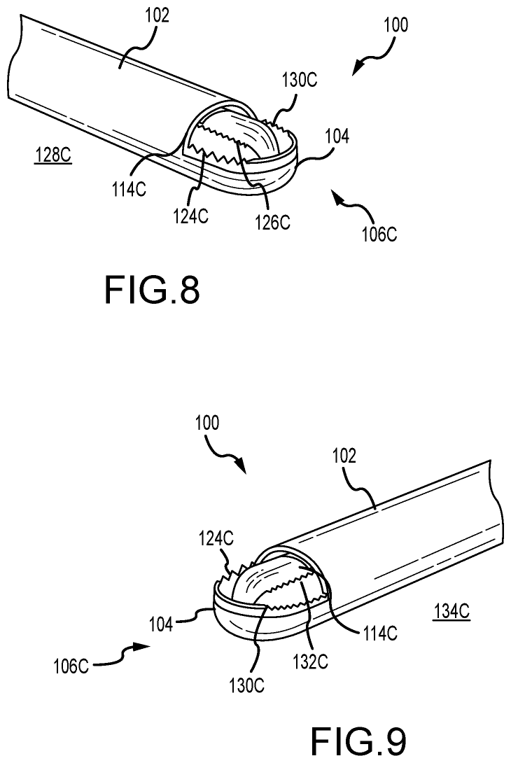

FIGS. 8-9 shows exemplary views of the distal end 106C and the keyhole 114C of the multi-blade 100, in accordance with aspects of the present disclosure. In FIGS. 8-9, the first side 128C of the distal end 106C includes an aggressive cutting edge 124C for the outer blade 102 and a smooth cutting edge 126C for the inner blade 104. In addition, the second side 134 of the distal end 106 includes smooth cutting edges 130C, 132C.

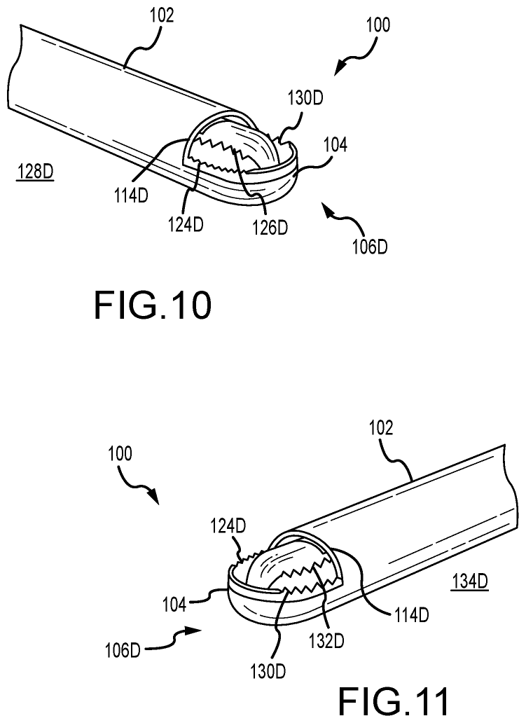

FIGS. 10-11 shows exemplary views of the distal end 106D and the keyhole 114D of the multi-blade 100, in accordance with aspects of the present disclosure. In FIGS. 10-11, the first side 128D of the distal end 106D includes a smooth cutting edge 124D for the outer blade 102 and an aggressive cutting edge 126D for the inner blade 104. In addition, the second side 134D of the distal end 106D includes aggressive cutting edges 130D, 132D.

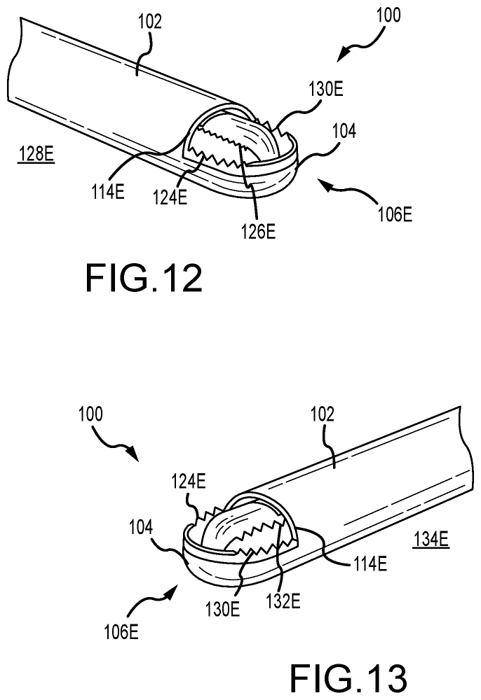

FIGS. 12-13 shows exemplary views of the distal end 106E and the keyhole 114E of the multi-blade 100, in accordance with aspects of the present disclosure. In FIGS. 12-13, the first side 128E of the distal end 106E includes an aggressive cutting edge 124E for the outer blade 102 and a smooth cutting edge 126E for the inner blade 104. In addition, the second side 134E of the distal end 106E includes aggressive cutting edges 130E, 132E.

It is noted the cutting edges 124, 126, 130, 132 are not limited to the smooth or aggressive patterns as shown in FIGS. 1A-13, but may be any type of cutting pattern as described throughout the present disclosure. In addition, it is noted the sides 128, 134 of the distal end 106 and corresponding edges 124, 126 and 130, 132 are not limited to what is shown in FIGS. 1A-13, and may be swapped without departing from the present disclosure. For example, the edges as described may be swapped between the first side 128 and the second side 134 of the distal end 106. The side-swapped edges may be beneficial to address issues with handedness (e.g., a right-handed tool versus a left-handed tool). In addition, the side-swapped edges may be beneficial in instances where access would render it difficult to effect a particular direction of cut, inhibiting the use of a particular cutting type or edge.

Although FIGS. 1A-1H and 1I-1P show cutting types or edges that are similar to FIGS. 4-5, it is noted the distal end 106 of the multi-blade 100 may be configured with the one or more bends or bent sections 112 from FIG. 2, the protrusion section 116 of the outer blade 102 with the exposed portion 120 of the inner blade 104 from FIG. 3, and/or any of the cutting types or edges shown in FIGS. 6-7, 8-9, 10-11, 12-13 without departing from the scope of the present disclosure.

Although FIGS. 2 and 3 show cutting types or edges that are similar to FIGS. 4-5, it is noted the distal end 106 of the multi-blade 100 may be configured with the one or more bends or bent sections 112 from FIG. 2, the protrusion section 116 of the outer blade 102 with the exposed portion 120 of the inner blade 104 from FIG. 3, and/or any of the cutting types or edges shown in FIGS. 6-7, 8-9, 10-11, 12-13 without departing from the scope of the present disclosure.

As generally depicted in FIGS. 1A-13, the inner blade 104 rotates within the interior cavity 104 with respect to the outer blade 102. Depending on the direction of rotation, a different type of cut may be effected by the multi-blade 100 on the tissue (e.g., soft tissue such as cartilage, osseous tissue such as bone, or the like) to be removed from the patient. Where the cutting edges are different depending on the side 128, 134 of the multi-blade 100, rotation in a first direction may effect a first type of cut, while rotation in a second direction may effect a second, different type of cut.

In one non-limiting example as illustrated in FIGS. 1A-1H, 1I-1P, and 4-5, rotation of the inner blade 104 in a counter-clockwise direction or CCW direction (e.g., as viewed from the distal end 106 of the multi-blade 100, in FIG. 1G) may effect an aggressive type of cut, due to the positioning of an aggressive cut pattern on the first cutting edge 124 of the outer blade 102 and the first cutting edge 126 of the inner blade 104. In addition, as illustrated in FIGS. 1A-1H, 1I-1P, and 4-5, rotation of the inner blade 104 in a clockwise direction or CW direction (e.g., as viewed from the distal end 106 of the multi-blade 100, in FIG. 1G) may effect a smooth type of cut, due to the positioning of a smooth cut pattern on the second cutting edge 130 of the outer blade 102 and the second cutting edge 132 of the inner blade 104.

In additional non-limiting examples as illustrated in FIGS. 6-7, 8-9, 10-11, and 12-13, varying the cutting edges or matching different cutting edges between the outer blade 102 and the inner blade 104 may result in a range of different cuts and/or amount of tissue removal. For example, combining a smooth cut and an aggressive cut between corresponding cutting edges on one side of the distal end 106 may result in an amount of tissue being removed that is less than a double-aggressive side depicted in FIGS. 4-5, and/or may result in an amount of tissue being removed that is more than a double-smooth side depicted in FIGS. 4-5.

Although aspects of the present disclosure are directed to cutting types or edges being configured relative to a particular direction of rotation, it is noted the direction of rotation may be different from that described above without departing from the scope of the present disclosure. For example, the alternative direction of rotation may be beneficial to address issues with handedness (e.g., a right-handed tool versus a left-handed tool). In addition, the alternative direction of rotation may be beneficial in instances where access would render it difficult to effect a particular direction of rotation, inhibiting the use of a particular cutting type or edge.

In some aspects, the inner blade 104 is capable of oscillation within the outer blade 102. Where the inner blade 104 oscillates, the amount of tissue removed may depend on the corresponding cutting edges of a particular side of the multi-blade 100 in contact with the tissue to be removed. The amount of tissue removed may additionally depend on one or more additional factors including, but not limited to, oscillation speed or frequency, pressure applied by a user (e.g., a surgeon), or other factors. For example, an increased application of pressure may result is a more aggressive amount of tissue being removed.

It is contemplated the outer blade 102 and/or the inner blade 104 may be fabricated through the use of any fabrication technique including, but not limited to, casting processes (die casting, injection molding, or the like), forming processes (e.g., extrusion, forging, stamping, additive manufacturing processes such as three-dimensional (3D) printing, or the like), cutting processes, joining processes, or the like. It is noted the fabrication processes may occur simultaneously, near-simultaneously, or sequentially where appropriate. For example, the outer blade 102 and the inner blade 104 may be simultaneously formed through an additive manufacturing process (with support material removed to allow the rotation of the inner blade 104 relative to the outer blade 102 where support material is used). In addition, it is noted the fabrication processes may occur separately on unique portions of the outer blade 102 and/or the inner blade 104 (e.g., a bend unique to a particular location on a component, or the like), and/or may be applied to the same portion of the outer blade 102 and/or the inner blade 104.

FIG. 14 generally shows an exemplary view of a medical shaver system 300, in accordance with aspects of the present disclosure. The medical shaver system 300 includes the medical shaver 200 with shaver handpiece 202 and multi-blade 100, and the control system 302. The multi-blade 100 shown in FIG. 14 may include any configuration as depicted and described in FIGS. 1A-13, without departing from the scope of the present disclosure. It is contemplated that any distal end 106 configuration of the multi-blade 100 may be dependent on the configuration of the shaver handpiece 202.

The multi-blade 100 is couplable to the shaver handpiece 202. For example, a working length of the multi-blade 100 ranges from 70 mm (2.756 in) to 180 mm (7.086 inches). In some aspects, the multi-blade 100 is attachable to and detachable from the shaver handpiece 202, such as where the multi-blade 100 is disposable or reusable, and the shaver handpiece 202 is reusable.

The shaver handpiece 202 includes a body 204 and a coupling mechanism 206. The multi-blade 100 is insertable into the coupling mechanism 206. For example, the coupling mechanism 206 at a distal end of the body 204 is operable to couple to a blade end 208 at the proximal end of the multi-blade 100, securing the multi-blade 100 to the shaver handpiece 202. It is noted the outer blade 102 and the inner blade 104 may be configured to engage the blade end 208 in such a way that the outer blade 102 is fixed in position or is capable of being manually rotated with respect to at least a portion of the blade end 208 and the inner blade 104 is rotatable via the at least a portion of the blade end 208. In addition, it is noted the blade end 208 may include rotatable components that allow for the transfer of power from an actuator or motor within the shaver handpiece 202 to the inner blade 104, causing the inner blade 104 to rotate relative to the outer blade 102.

In one non-limiting example, the blade end 208 is insertable into the coupling mechanism 206, and a blade collar 210 of the coupling mechanism 206 is operable to slide in a direction along a coaxial central axis shared by the multi-blade 100 and the coupling mechanism 206 to engage and retain the multi-blade 100 in the coupling mechanism 206. It is noted the multi-blade 100 may be retained within the coupling mechanism 206 in one or more positions. For example, the coupling mechanism 206 and the blade end 208 may include interlocking recesses and protrusions that allow for the positioning of the multi-blade 100 within the coupling mechanism 206 at one or multiple orientations from a defined 0 degrees (e.g., 0°, 90°, 180°, 270°, or the like), where 0 degrees is defined as pointing at an upper surface of the body 204. In addition to, or instead of, being able to position the entire multi-blade 100 within the coupling mechanism 206 at one or more multiple orientations, the outer blade 102 may be capable of being manually rotated to a desired orientation ranging from 0° to 360°. The ability to orientate the multi-blade 100 within the coupling mechanism 206 and/or the ability to manually rotate and orientate the outer blade 102 allows for the positioning of the keyhole window 114 in multiple directions, to engage with tissue for removal in any direction as desired. In general, however, it is noted the outer blade 102 may be capable of being manually rotated with respect to the blade end 208, or may be at a fixed orientation, without departing from the scope of the present disclosure.

The shaver handpiece 202 includes a suction collar 212 with a toggle 214. The toggle 214 may include a lever, button, switch, or other device operable to increase or decrease an amount of suction through the shaver handpiece 202 and into the multi-blade 100 based on a received user input. The body 204 includes a suction channel that is in fluid communication with the interior cavity 110 of the multi-blade 100 at the distal end of the body 204 and with a suction tube 216 at a proximal end of the body 204. In some aspects, the suction collar 212 may include a locking mechanism 218 that allows for removal of the suction collar 212 to clean any tissue removed by the multi-blade 100 from the suction channel through the body 204 of the shaver handpiece 202. It should be understood the blade end 208 includes any internal components as necessary to transfer suction from the suction channel through the body 204 to the multi-blade 100 (e.g., through the interior cavity 111 defined within the inner blade 104, and/or through the interior cavity 110 within the outer walls 108 of the outer blade 102 and surrounding the inner blade 104).

The shaver handpiece 202 includes one or more toggles 220 on the body 204 that provide a user with control of one or more of rotation (including speed of rotation and/or direction of rotation) and/or oscillation (including speed of oscillation) of the inner blade 104 relative to the outer blade 102. For instance, the speeds may be pre-set for a particular toggle 220, or may be gradually increased or decreased with the one or more toggles 220.

In some aspects, the medical shaver 200 is in communication with the control system 302. In some aspects, wires 304 couple the medical shaver 200 to the control system 302, and provide control signals and/or power between the medical shaver 200 and the control system 302. It should be understood that the blade end 208 may include mechanical internal components (e.g., couplers, bearing assemblies, gearing assemblies, or the like) and/or electrical internal components (e.g., circuitry or sensors operable to transfer power and/or data or electrical signals from the wires 304 to the inner blade 104) capable of transferring and/or monitoring rotational motion from the shaver handpiece 202 to the inner blade 104.

In one non-limiting example, user inputs entered via one or more user input devices 306 on the control system 302 may be transmitted to the medical shaver 200 to control one or more of rotation (including speed of rotation and/or direction of rotation) and/or oscillation (including speed of oscillation) of the inner blade 104 relative to the outer blade 102. For instance, activation of a first user input device 306 on the control system 302 may control rotation in a first direction (e.g., a CW direction), and activation of a second user input device 306 on the control system 302 may control rotation in a second, different direction (e.g., a CCW direction). In addition, activation of at least a third user input device 306 may control speed of rotation in the first direction and/or the second, different direction. In another non-limiting example, power may be transmitted to the medical shaver 200 (e.g., via the wires 304, or wirelessly). The one or more toggles 220 on the body 204 may provide a user with control of one or more of rotation (including speed of rotation and/or direction of rotation) and/or oscillation (including speed of oscillation) of the inner blade 104 relative to the outer blade 102. For instance, activation of a first toggle 220 on the body 204 may control rotation in a first direction (e.g., a CW direction), and activation of a second toggle 220 on the body 204 may control rotation in a second, different direction (e.g., a CCW direction). In this example, data or electrical signals may additionally be transmitted to the control system 302 (e.g., via the wires 304, or wirelessly), and an output device 308 (e.g., a visual output device such as a display or lights, aural output device such as a speaker or microphone, or the like) may indicate the speed of rotation, direction of rotation, and/or speed of oscillation. Where the output device 308 is a touchscreen display, the one or more user input devices 306 may be membrane switches or touch sensor embedded within a film over a screen or positioned between a film and the screen.

Although aspects of the present disclosure are directed to the multi-blade 100 being attachable to and detachable from the shaver handpiece 202, it is noted the multi-blade 100 may be fixedly coupled to the shaver handpiece 202 without departing from the scope of the present disclosure. Here, the entire medical shaver 200 may be considered disposable or reusable.

Operation of the medical shaver 200 with the multi-blade 100 may include one or more of the following steps.

In a step, a direction and/or speed of rotation of the inner blade 104 is determined via toggles 220 on the shaver handpiece 202 and/or via user inputs on the control system 302.

In a step, a rotational motion is transmitted to the inner blade 104 via the shaver handpiece 202 that corresponds to the determined direction and/or speed of rotation.

In a step, rotation of the inner blade 104 in a first direction causes a removal of a first amount of tissue. In another step, rotation of the inner blade 104 in a second direction causes a removal of a second amount of material. The first amount of material may be the same as, less than, or greater than the second amount of material depending on the type of cutting edges on the inner blade 104. In one non-limiting example, side 128 includes an aggressive-type first cutting edge 124 of the outer blade 102 and an aggressive-type first cutting edge 126 of the inner blade 104, and rotation in a CCW direction removes a first amount of material. In this example, side 134 includes a smooth-type second cutting edge 130 of the outer blade 102 and a smooth-type second cutting edge of the inner blade 104, and rotation in a CW direction removes a second amount of material. Due to the difference in the aggressive cutting edges 124, 126 versus the smooth cutting edges 130, 132, the first amount of material removed is greater than the second amount of material removed.

In a step, the first amount of material and/or the second amount of material is collected within the interior cavity 110 defined by the outer blade 102 during or following removal from the patient.

In a step, an amount of suction force is provided through the multi-blade 100 (e.g., through the interior cavity 111 defined within the inner blade 104, and/or through the interior cavity 110 within the outer walls 108 of the outer blade 102 and surrounding the inner blade 104) to remove the first amount of material and/or the second amount of material from the interior cavity 110 through the multi-blade 100 and the shaver handpiece 202.

Any of the steps, functions, and operations discussed herein can be performed continuously and automatically.

While the steps have been discussed and illustrated in relation to a particular sequence of events, it should be appreciated that changes, additions, and omissions to this sequence can occur without materially affecting the operation of the disclosed embodiments, configuration, and aspects.

The exemplary systems and methods of this disclosure have been described in relation to the multi-blade 100, the medical shaver 200 including the multi-blade 100 and the shaver handpiece 202, and/or the control system 302. However, to avoid unnecessarily obscuring the present disclosure, the preceding description may omit a number of known structures and devices. This omission is not to be construed as a limitation of the scope of the claimed disclosure. Specific details are set forth to provide an understanding of the present disclosure. It should, however, be appreciated that the present disclosure may be practiced in a variety of ways beyond the specific detail set forth herein.

A number of variations and modifications of the disclosure can be used. It would be possible to provide for some features of the disclosure without providing others. In addition, it would be possible to combine some features of the disclosure without combining all.

References in the specification to “being operable” or “is operable” may be understood as “being configured to” or “is configured to, “being capable of” or “is capable of,” and the like.

References in the specification to “an aspect,” “one embodiment,” “an embodiment,” “an example embodiment,” “some embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in conjunction with one embodiment, it is submitted that the description of such feature, structure, or characteristic may apply to any other embodiment unless so stated and/or except as will be readily apparent to one skilled in the art from the description. The present disclosure, in various embodiments, configurations, and aspects, includes components, methods, processes, systems and/or apparatus substantially as depicted and described herein, including various embodiments, subcombinations, and subsets thereof. Those of skill in the art will understand how to make and use the systems and methods disclosed herein after understanding the present disclosure. The present disclosure, in various embodiments, configurations, and aspects, includes providing devices and processes in the absence of items not depicted and/or described herein or in various embodiments, configurations, or aspects hereof, including in the absence of such items as may have been used in previous devices or processes, e.g., for improving performance, achieving ease, and/or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments, configurations, or aspects for the purpose of streamlining the disclosure. The features of the embodiments, configurations, or aspects of the disclosure may be combined in alternate embodiments, configurations, or aspects other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment, configuration, or aspect. Thus, the following claims are hereby incorporated into this Detailed Description, with each claim standing on its own as a separate preferred embodiment of the disclosure.

Moreover, though the description of the disclosure has included description of one or more embodiments, configurations, or aspects and certain variations and modifications, other variations, combinations, and modifications are within the scope of the disclosure, e.g., as may be within the skill and knowledge of those in the art, after understanding the present disclosure. It is intended to obtain rights, which include alternative embodiments, configurations, or aspects to the extent permitted, including alternate, interchangeable and/or equivalent structures, functions, ranges, or steps to those claimed, whether or not such alternate, interchangeable and/or equivalent structures, functions, ranges, or steps are disclosed herein, and without intending to publicly dedicate any patentable subject matter.

Exemplary aspects are directed toward:
A multi-blade for a medical shaver, comprising:
an outer blade including one or more walls defining an interior cavity, wherein the outer blade includes a keyhole in the one or more walls at a distal end of the multi-blade; and
an inner blade housed within the interior cavity, wherein the inner blade is operable to rotate relative to the outer blade, and wherein at least a portion of the inner blade is exposed via the keyhole,
wherein a first side of the keyhole includes a first cutting edge on the outer blade and a corresponding first cutting edge on the inner blade,
wherein a second side of the keyhole includes a second cutting edge on the outer blade and a corresponding second cutting edge on the inner blade, and
wherein the second cutting edge is different from the first cutting edge.

Any one or more of the above aspects include wherein rotation of the inner blade in a first direction causes the first cutting edge on the outer blade and the corresponding first cutting edge on the inner blade to engage and remove a first amount of tissue from a patient, and wherein rotation of the inner blade in a second, different direction causes the second cutting edge on the outer blade and the corresponding second cutting edge on the inner blade to engage and remove a second, different amount of tissue from the patient.

Any one or more of the above aspects include wherein the first cutting edges of the outer blade and the inner blade are each an aggressive cut, wherein the second cutting edges of the outer blade and the inner blade are each a smooth cut, and wherein the first amount of tissue removed is greater than the second amount of tissue removed via rotation of the inner blade.

Any one or more of the above aspects include wherein the first cutting edges of the outer blade and the inner blade are each a smooth cut, wherein the second cutting edges of the outer blade and the inner blade are each an aggressive cut, and wherein the first amount of tissue removed is less than the second amount of tissue removed via rotation of the inner blade.

Any one or more of the above aspects include wherein at least one of the first cutting edge of the outer blade and the first cutting edge of the inner blade on the first side of the keyhole is an aggressive cut, and wherein at least one of the second cutting edge of the outer blade and the second cutting edge of the inner blade on the second side of the keyhole is a smooth cut.

Any one or more of the above aspects include wherein at least one of the first cutting edge of the outer blade and the first cutting edge of the inner blade on the first side of the keyhole is a smooth cut, and wherein at least one of the second cutting edge of the outer blade and the second cutting edge of the inner blade on the second side of the keyhole is an aggressive cut.

Any one or more of the above aspects include the multi-blade further comprising one or more bends along a length of the multi-blade between the distal end and a proximal end of the multi-blade.

Any one or more of the above aspects include the multi-blade further comprising a blade end at a proximal end of the multi-blade, wherein the blade end is couplable to a distal end of a shaver handpiece of the medical shaver.

Any one or more of the above aspects include wherein the outer blade is fixed in position or is capable of being manually rotated relative to the shaver handpiece of the medical shaver via the blade end, and wherein the inner blade is operable to rotate relative to the shaver handpiece of the medical shaver via a transfer of rotational motion through the blade end.

Exemplary aspects are directed toward:
A medical shaver, comprising:
a multi-blade, the multi-blade comprising:
an outer blade including one or more walls defining an interior cavity, wherein the outer blade includes a keyhole in the one or more walls at a distal end of the multi-blade; and
an inner blade housed within the interior cavity, wherein the inner blade is operable to rotate relative to the outer blade, and wherein at least a portion of the inner blade is exposed via the keyhole,
wherein a first side of the keyhole includes a first cutting edge on the outer blade and a corresponding first cutting edge on the inner blade,
wherein a second side of the keyhole includes a second cutting edge on the outer blade and a corresponding second cutting edge on the inner blade, and
wherein the second cutting edge is different from the first cutting edge; and a shaver handpiece comprising a body and a coupling mechanism at a distal end of the shaver handpiece,
wherein a proximal end of the multi-blade is insertable into the coupling mechanism and securable to the shaver handpiece via the coupling mechanism.

Any one or more of the above aspects include wherein the multi-blade further comprises a blade end at the proximal end of the multi-blade, wherein the blade end is insertable into the coupling mechanism to secure the multi-blade to the shaver handpiece.

Any one or more of the above aspects include wherein the outer blade is fixed in position or is capable of being manually rotated relative to the shaver handpiece of the medical shaver via the blade end, and wherein the inner blade is operable to rotate relative to the shaver handpiece of the medical shaver via a transfer of rotational motion through the blade end.

Any one or more of the above aspects include wherein rotation of the inner blade in a first direction causes the first cutting edge on the outer blade and the corresponding first cutting edge on the inner blade to engage and remove a first amount of tissue from a patient, and wherein rotation of the inner blade in a second, different direction causes the second cutting edge on the outer blade and the corresponding second cutting edge on the inner blade to engage and remove a second, different amount of tissue from the patient.

Any one or more of the above aspects include wherein the first cutting edges of the outer blade and the inner blade are each an aggressive cut, wherein the second cutting edges of the outer blade and the inner blade are each a smooth cut, and wherein the first amount of tissue removed is greater than the second amount of tissue removed via rotation of the inner blade.

Any one or more of the above aspects include wherein the interior cavity defined by the one or more walls of the outer blade is operable to collect one or more of the removed first amount of tissue or the removed second amount of tissue.

Any one or more of the above aspects include wherein the first amount of tissue and/or the second amount of tissue collected within the interior cavity is removable from the multi-blade via application of a suction force provided through the multi-blade.

Exemplary aspects are directed toward:
A medical shaver system, comprising:
a medical shaver, the medical shaver comprising:
a multi-blade, the multi-blade comprising:
an outer blade including one or more walls defining an interior cavity, wherein the outer blade includes a keyhole in the one or more walls at a distal end of the multi-blade; and
an inner blade housed within the interior cavity, wherein the inner blade is operable to rotate relative to the outer blade, and wherein at least a portion of the inner blade is exposed via the keyhole,
wherein a first side of the keyhole includes a first cutting edge on the outer blade and a corresponding first cutting edge on the inner blade,
wherein a second side of the keyhole includes a second cutting edge on the outer blade and a corresponding second cutting edge on the inner blade, and
wherein the second cutting edge is different from the first cutting edge; and a shaver handpiece comprising a body and a coupling mechanism at a distal end of the shaver handpiece,
wherein a proximal end of the multi-blade is insertable into the coupling mechanism and securable to the shaver handpiece via the coupling mechanism; and
a control system in communication with the medical shaver, wherein the control system is operable to display at least one of direction of rotation and speed of rotation of the inner blade.

Any one or more of the above aspects include wherein the at least one of direction of rotation and speed of rotation of the inner blade is controllable via at least one of one or more user inputs on the control system and/or one or more toggles on the shaver handpiece.

Any one or more of the above aspects include wherein activation of a first user input on the control system or a first toggle on the shaver handpiece causes the inner blade to rotate in the first direction, and wherein activation of a second user input on the control system or a second toggle on the shaver handpiece causes the inner blade to rotate in a second, different direction.

Any one or more of the above aspects include wherein rotation of the inner blade in a first direction causes the first cutting edge on the outer blade and the corresponding first cutting edge on the inner blade to engage and remove a first amount of tissue from a patient, and wherein rotation of the inner blade in a second, different direction causes the second cutting edge on the outer blade and the corresponding second cutting edge on the inner blade to engage and remove a second, different amount of tissue from the patient.

Any one or more of the above aspects/embodiments as substantially disclosed herein.

Any one or more of the aspects/embodiments as substantially disclosed herein optionally in combination with any one or more other aspects/embodiments as substantially disclosed herein.

One or means adapted to perform any one or more of the above aspects/embodiments as substantially disclosed herein.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

As used herein, the singular forms “a,” “an,” and “the” are intended to include the plural forms as well, unless the context clearly indicates otherwise. It will be further understood that the terms “include,” “including,” “includes,” “comprise,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof. The term “and/or” includes any and all combinations of one or more of the associated listed items. The use of “including,” “comprising,” or “having” and variations thereof herein is meant to encompass the items listed thereafter and equivalents thereof as well as additional items. Accordingly, the terms “including,” “comprising,” or “having” and variations thereof can be used interchangeably herein.

The transitional phrase “consisting of” excludes any element, step, or ingredient not specified in the claim, but does not exclude additional components or steps that are unrelated to the disclosure such as impurities ordinarily associated therewith. The transitional phrase “consisting essentially of” limits the scope of a claim to the specified materials or steps and those that do not materially affect the basic and novel characteristic(s) of the claimed invention.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” and “A, B, and/or C” means A alone, B alone, C alone, A and B together, A and C together, B and C together, or A, B, and C together. When each one of A, B, and C in the above expressions refers to an element, such as X, Y, and Z, or a class of elements, such as X₁-Xₙ, Y₁-Yₘ, and Z₁-Zₒ, the phrase is intended to refer to a single element selected from X, Y, and Z, a combination of elements selected from the same class (e.g., X₁ and X₂) as well as a combination of elements selected from two or more classes (e.g., Y₁ and Zₒ).

The term “automatic” and variations thereof, as used herein, refers to any process or operation, which is typically continuous or semi-continuous, done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

The terms “determine,” “calculate,” “compute,” and variations thereof, as used herein, are used interchangeably and include any type of methodology, process, mathematical operation, or technique.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and this disclosure.

Unless otherwise indicated, all numbers expressing quantities, dimensions, conditions, ratios, ranges, and so forth used in the specification and claims are to be understood as being modified in all instances by the term “about” or “approximately.” Accordingly, unless otherwise indicated, all numbers expressing quantities, dimensions, conditions, ratios, ranges, and so forth used in the specification and claims may be increased or decreased by approximately 5% to achieve satisfactory results. Additionally, where the meaning of the terms “about” or “approximately” as used herein would not otherwise be apparent to one of ordinary skill in the art, the terms “about” and “approximately” should be interpreted as meaning within plus or minus 5% of the stated value.

All ranges described herein may be reduced to any sub-range or portion of the range, or to any value within the range without deviating from the invention. For example, the range “5 to 55” includes, but is not limited to, the sub-ranges “5 to 20” as well as “17 to 54.”

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

What is claimed is:

1. A multi-blade for a medical shaver, comprising:
an outer blade including one or more walls defining an interior cavity, wherein the outer blade includes a keyhole in the one or more walls at a distal end of the multi-blade; and
an inner blade housed within the interior cavity, wherein the inner blade is operable to rotate relative to the outer blade, and wherein at least a portion of the inner blade is exposed via the keyhole,
wherein a first side of the keyhole includes a first cutting edge on the outer blade and a corresponding first cutting edge on the inner blade,
wherein a second side of the keyhole includes a second cutting edge on the outer blade and a corresponding second cutting edge on the inner blade, and
wherein the second cutting edge is different from the first cutting edge.

2. The multi-blade of claim 1, wherein rotation of the inner blade in a first direction causes the first cutting edge on the outer blade and the corresponding first cutting edge on the inner blade to engage and remove a first amount of tissue from a patient, and wherein rotation of the inner blade in a second, different direction causes the second cutting edge on the outer blade and the corresponding second cutting edge on the inner blade to engage and remove a second, different amount of tissue from the patient.

3. The multi-blade of claim 2, wherein the first cutting edges of the outer blade and the inner blade are each an aggressive cut, wherein the second cutting edges of the outer blade and the inner blade are each a smooth cut, and wherein the first amount of tissue removed is greater than the second amount of tissue removed via rotation of the inner blade.

4. The multi-blade of claim 3, wherein the first cutting edges of the outer blade and the inner blade are each a smooth cut, wherein the second cutting edges of the outer blade and the inner blade are each an aggressive cut, and wherein the first amount of tissue removed is less than the second amount of tissue removed via rotation of the inner blade.

5. The multi-blade of claim 1, wherein at least one of the first cutting edge of the outer blade and the first cutting edge of the inner blade on the first side of the keyhole is an aggressive cut, and wherein at least one of the second cutting edge of the outer blade and the second cutting edge of the inner blade on the second side of the keyhole is a smooth cut.

6. The multi-blade of claim 1, wherein at least one of the first cutting edge of the outer blade and the first cutting edge of the inner blade on the first side of the keyhole is a smooth cut, and wherein at least one of the second cutting edge of the outer blade and the second cutting edge of the inner blade on the second side of the keyhole is an aggressive cut.

7. The multi-blade of claim 1, the multi-blade further comprising one or more bends along a length of the multi-blade between the distal end and a proximal end of the multi-blade.

8. The multi-blade of claim 1, the multi-blade further comprising a blade end at a proximal end of the multi-blade, wherein the blade end is couplable to a distal end of a shaver handpiece of the medical shaver.

9. The multi-blade of claim 8, wherein the outer blade is fixed in position or is capable of being manually rotated relative to the shaver handpiece of the medical shaver via the blade end, and wherein the inner blade is operable to rotate relative to the shaver handpiece of the medical shaver via a transfer of rotational motion through the blade end.

10. A medical shaver, comprising:
a multi-blade, the multi-blade comprising:
an outer blade including one or more walls defining an interior cavity, wherein the outer blade includes a keyhole in the one or more walls at a distal end of the multi-blade; and
an inner blade housed within the interior cavity, wherein the inner blade is operable to rotate relative to the outer blade, and wherein at least a portion of the inner blade is exposed via the keyhole,
wherein a first side of the keyhole includes a first cutting edge on the outer blade and a corresponding first cutting edge on the inner blade,
wherein a second side of the keyhole includes a second cutting edge on the outer blade and a corresponding second cutting edge on the inner blade, and
wherein the second cutting edge is different from the first cutting edge; and
a shaver handpiece comprising a body and a coupling mechanism at a distal end of the shaver handpiece,
wherein a proximal end of the multi-blade is insertable into the coupling mechanism and securable to the shaver handpiece via the coupling mechanism.

11. The medical shaver of claim 10, wherein the multi-blade further comprises a blade end at the proximal end of the multi-blade, wherein the blade end is insertable into the coupling mechanism to secure the multi-blade to the shaver handpiece.

12. The medical shaver of claim 10, wherein the outer blade is fixed in position or is capable of being manually rotated relative to the shaver handpiece of the medical shaver via the blade end, and wherein the inner blade is operable to rotate relative to the shaver handpiece of the medical shaver via a transfer of rotational motion through the blade end.

13. The medical shaver of claim 10, wherein rotation of the inner blade in a first direction causes the first cutting edge on the outer blade and the corresponding first cutting edge on the inner blade to engage and remove a first amount of tissue from a patient, and wherein rotation of the inner blade in a second, different direction causes the second cutting edge on the outer blade and the corresponding second cutting edge on the inner blade to engage and remove a second, different amount of tissue from the patient.

14. The medical shaver of claim 13, wherein the first cutting edges of the outer blade and the inner blade are each an aggressive cut, wherein the second cutting edges of the outer blade and the inner blade are each a smooth cut, and wherein the first amount of tissue removed is greater than the second amount of tissue removed via rotation of the inner blade.

15. The medical shaver of claim 13, wherein the interior cavity defined by the one or more walls of the outer blade is operable to collect one or more of the removed first amount of tissue or the removed second amount of tissue.

16. The medical shaver of claim 15, wherein the first amount of tissue and/or the second amount of tissue collected within the interior cavity is removable from the multi-blade via application of a suction force provided through the multi-blade.

17. A medical shaver system, comprising:
a medical shaver, the medical shaver comprising:
a multi-blade, the multi-blade comprising:
an outer blade including one or more walls defining an interior cavity,
wherein the outer blade includes a keyhole in the one or more walls at a distal end of the multi-blade; and
an inner blade housed within the interior cavity, wherein the inner blade is operable to rotate relative to the outer blade, and wherein at least a portion of the inner blade is exposed via the keyhole,
wherein a first side of the keyhole includes a first cutting edge on the outer blade and a corresponding first cutting edge on the inner blade,
wherein a second side of the keyhole includes a second cutting edge on the outer blade and a corresponding second cutting edge on the inner blade, and
wherein the second cutting edge is different from the first cutting edge; and
a shaver handpiece comprising a body and a coupling mechanism at a distal end of the shaver handpiece,
wherein a proximal end of the multi-blade is insertable into the coupling mechanism and securable to the shaver handpiece via the coupling mechanism; and
a control system in communication with the medical shaver, wherein the control system is operable to display at least one of direction of rotation and speed of rotation of the inner blade.

18. The medical shaver system of claim 17, wherein the at least one of direction of rotation and speed of rotation of the inner blade is controllable via at least one of one or more user inputs on the control system and one or more toggles on the shaver handpiece.

19. The medical shaver system of claim 18, wherein activation of a first user input on the control system or a first toggle on the shaver handpiece causes the inner blade to rotate in a first direction, and wherein activation of a second user input on the control system or a second toggle on the shaver handpiece causes the inner blade to rotate in a second, different direction.

20. The medical shaver system of claim 17, wherein rotation of the inner blade in a first direction causes the first cutting edge on the outer blade and the corresponding first cutting edge on the inner blade to engage and remove a first amount of tissue from a patient, and wherein rotation of the inner blade in a second, different direction causes the second cutting edge on the outer blade and the corresponding second cutting edge on the inner blade to engage and remove a second, different amount of tissue from the patient.
